(19) United States
(12) Patent Application Publication
(10) Pub. No.: US 2026/0207358 A1
(43) Pub. Date: Jul. 23, 2026

(54) IMPLANTABLE MEDICAL DEVICE AND STENT DELIVERY SYSTEM

(71) Applicant: TERUMO KABUSHIKI KAISHA, Tokyo (JP)

(72) Inventors: Shuhei MATSUSHITA, Hadano Kanagawa (JP); Yuhei MORI, Yamato Kanagawa (JP); Yuji MOTOSE, Hiratsuka Kanagawa (JP); Tsutomu SUGIKI, Fuji Shizuoka (JP)

(21) Appl. No.: 19/571,383

(22) Filed: Mar. 18, 2026

Related U.S. Application Data
(63) Continuation of application No. PCT/JP2024/032872, filed on Sep. 13, 2024.

(30) Foreign Application Priority Data
Sep. 19, 2023 (JP) 2023-150811

(51) Int. Cl. A61F 2/89; A61F 2/958

(52) U.S. Cl. CPC A61F 2/89; A61F 2/958; A61F 2220/0058; A61F 2230/0004; A61F 2230/0069; A61F 2250/0067

(57) ABSTRACT

An implantable medical device includes a stent that has a cylindrical shape extending in an axial direction, is expandable in a radial direction, and includes: a plurality of wavy rings forming an outer periphery of the cylindrical shape, and a plurality of link portions each connecting two of the rings that are adjacent to each other; a porous structure that covers the stent and has a plurality of through-holes; and a plurality of first fixing portions by which at least part of the porous structure is fixed to the stent, each of the first fixing portions being located on the stent at a position other than distal and proximal ends of the stent.

### CROSS-REFERENCE TO RELATED APPLICATION

This application is a continuation of International Patent Application No. PCT/JP2024/032872 filed September 13, 2024, which is based upon and claims the benefit of priority from Japanese Patent Application No. 2023-150811, filed September 19, 2023, the entire contents of which are incorporated herein by reference.

### TECHNICAL FIELD

The present disclosure relates to an implantable medical device and a stent delivery system.

### BACKGROUND ART

A stent is a medical device that is delivered to a lesion in a lumen of a living body by a stent delivery system and then implanted or indwelled to expand the lesion such as a stenosed site or an occluded site and secure a passage thereof in order to treat various diseases caused by stenosis or occlusion of the lumen such as a blood vessel. The stent has wire-like rings forming a cylindrical outer periphery at which gaps are formed, and a plurality of link portions connecting the rings at the gaps.

For example, there is a known stent device in which a porous structure is disposed on a surface of a support element in order to prevent peripheral emboli when a stent is placed. In the stent device, the porous structure expands following expansion of the support element, so that it is possible to prevent scattering of a thrombus accompanying expansion of the support element.

### SUMMARY

In such a stent device, the porous structure is fixed to the support element only at both ends of the support element, and thus, a portion (portion excluding both ends) of the porous structure that is not fixed to the support element is deformed so as to spread radially outward at the time of clinical use, and the outer diameter becomes large, which may cause deterioration in deliverability of the stent device.

Embodiments of the present disclosure provide an implantable medical device and a stent delivery system capable of suppressing deterioration in deliverability due to a porous structure being deformed to spread radially outward.

In one embodiment, an implantable medical device comprises: a stent that has a cylindrical shape extending in an axial direction, is expandable in a radial direction, and includes: a plurality of wavy rings forming an outer periphery of the cylindrical shape, and a plurality of link portions each connecting two of the rings that are adjacent to each other; a porous structure that covers the stent and has a plurality of through-holes; and a plurality of first fixing portions by which at least part of the porous structure is fixed to the stent, each of the first fixing portions being located on the stent at a position other than distal and proximal ends of the stent.

With this configuration, at least part of the porous structure is fixed to the stent at a position excluding the distal end and the proximal end by the fixing portion, so that it is possible to suppress the porous structure from being deformed so as to spread radially outward and an outer diameter of the entire implantable medical device from becoming large. It is therefore possible to provide the implantable medical device capable of suppressing deterioration in deliverability due to the porous structure being deformed so as to spread radially outward.

### BRIEF DESCRIPTION OF DRAWINGS

FIG. 1 is a schematic plan view illustrating a stent delivery system including an implantable medical device according to an embodiment.

FIG. 2 is a schematic plan view illustrating a contracted state of the implantable medical device.

FIG. 3 is a schematic plan view illustrating an expanded state of the implantable medical device.

FIG. 4 is a partially enlarged view of the implantable medical device.

FIG. 5 is a partially enlarged view of a portion A in FIG. 4.

FIG. 6 is a partially enlarged view of a portion A in FIG. 5.

FIG. 7A is a cross-sectional view taken along a line 7A-7A in FIG. 6.

FIG. 7B is a cross-sectional view taken along a line 7B-7B in FIG. 6.

FIG. 8 is a schematic plan view illustrating the vicinity of a distal end of the implantable medical device.

FIG. 9 is a partially enlarged view illustrating a portion A in FIG. 8.

FIG. 10 illustrates a shape of a stent in a contracted state with a two-dot chain line and illustrates a shape of the stent in an expanded state with a solid line.

FIG. 11 is a view corresponding to FIG. 4 illustrating an implantable medical device according to a modification.

FIG. 12 is a schematic view illustrating a stent according to a modification.

FIG. 13 is a schematic view illustrating a stent according to a modification.

### DETAILED DESCRIPTION

Embodiments of the present disclosure will be described below with reference to the accompanying drawings. Note that the following description does not limit the technical scope or the significance of each term disclosed in the claims. The dimensional ratios in the drawings are exaggerated for convenience of illustration and may be different from actual ratios.

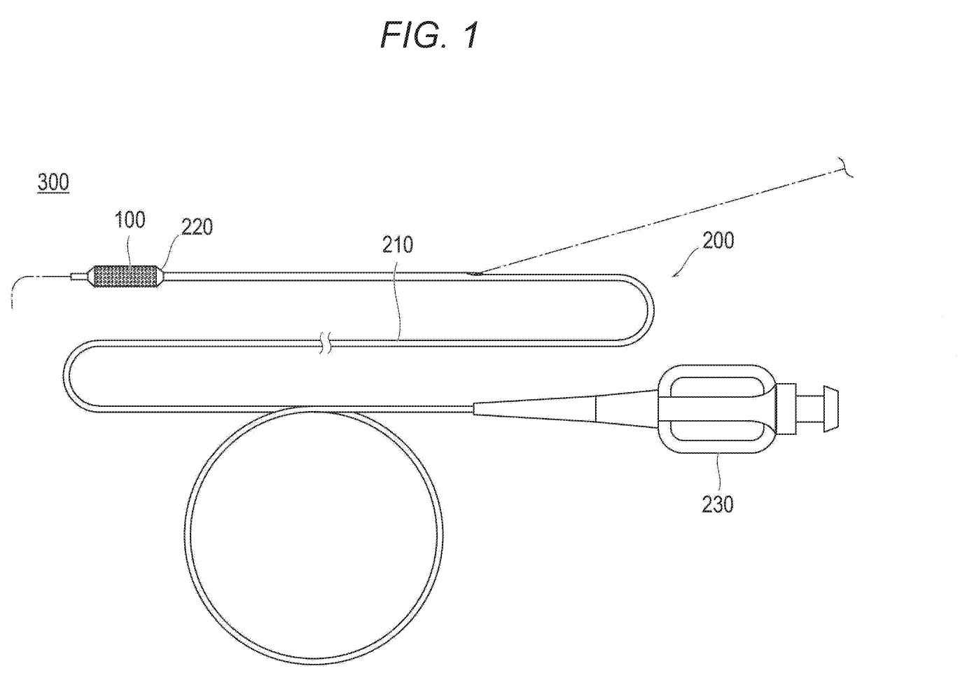

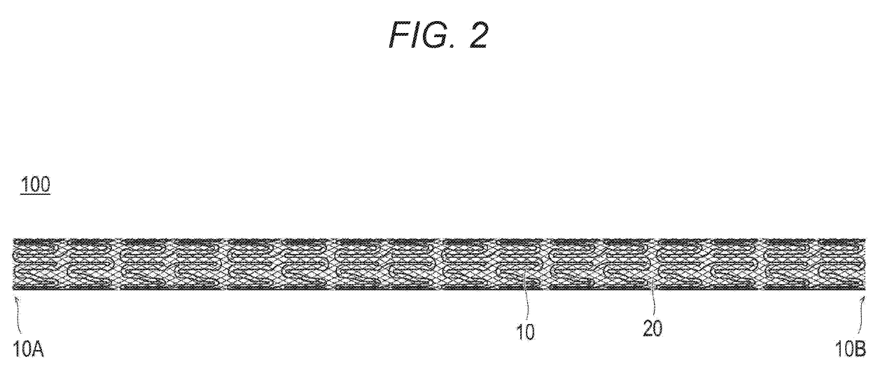

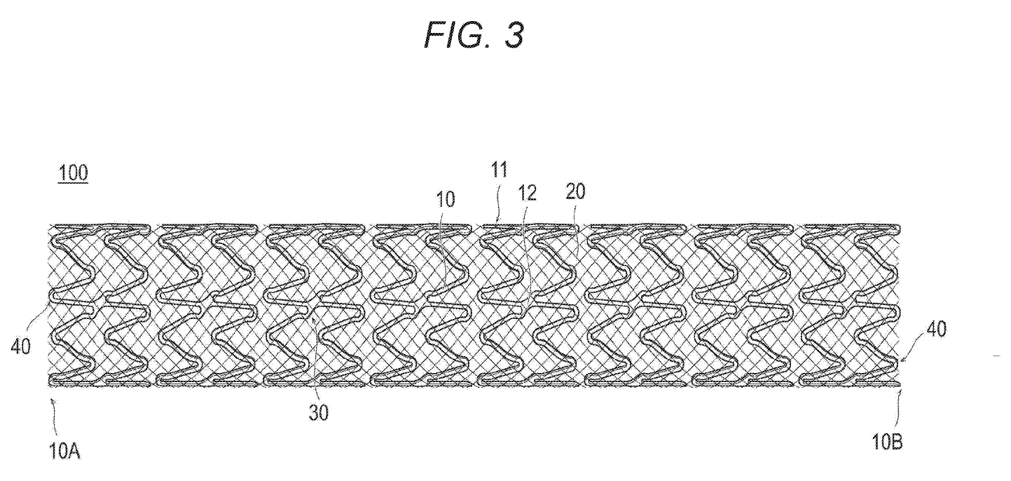

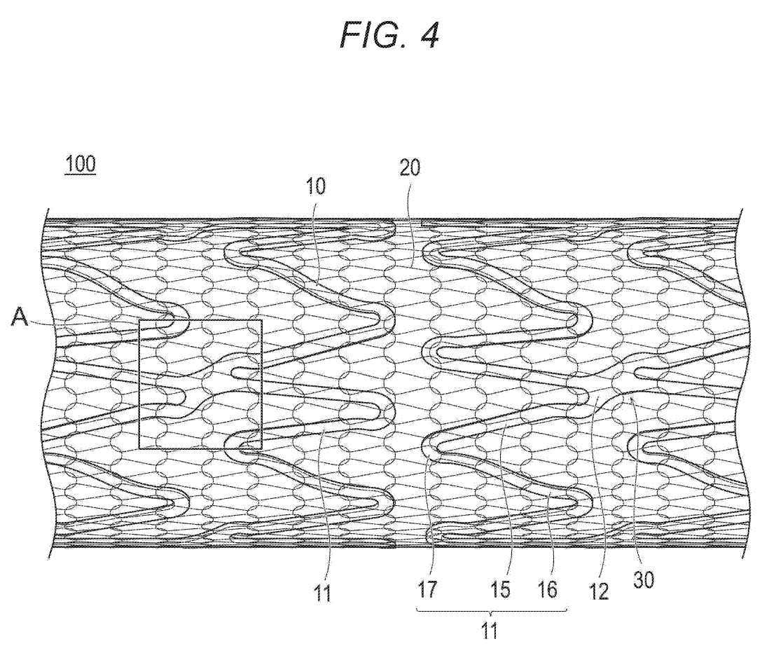

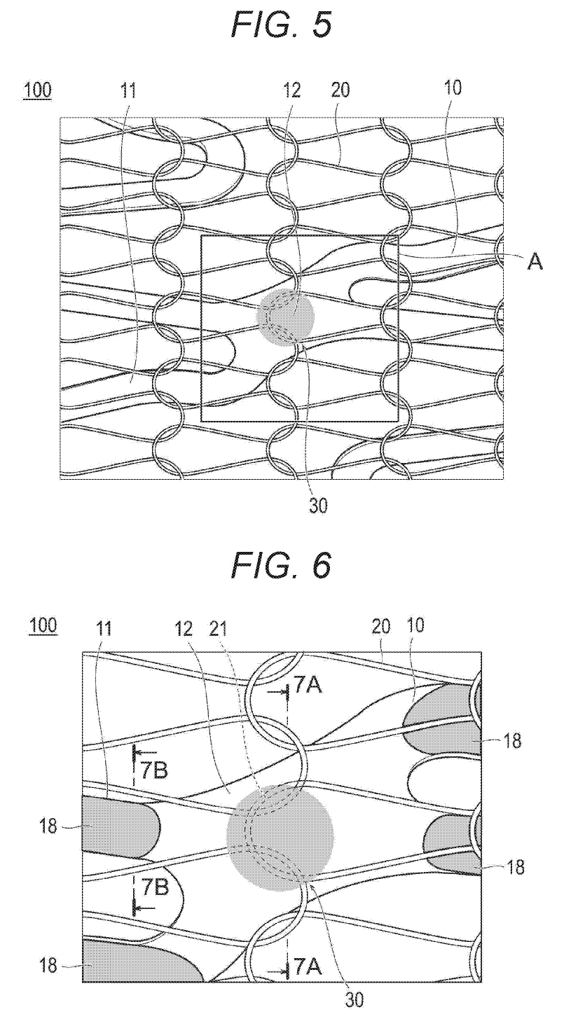

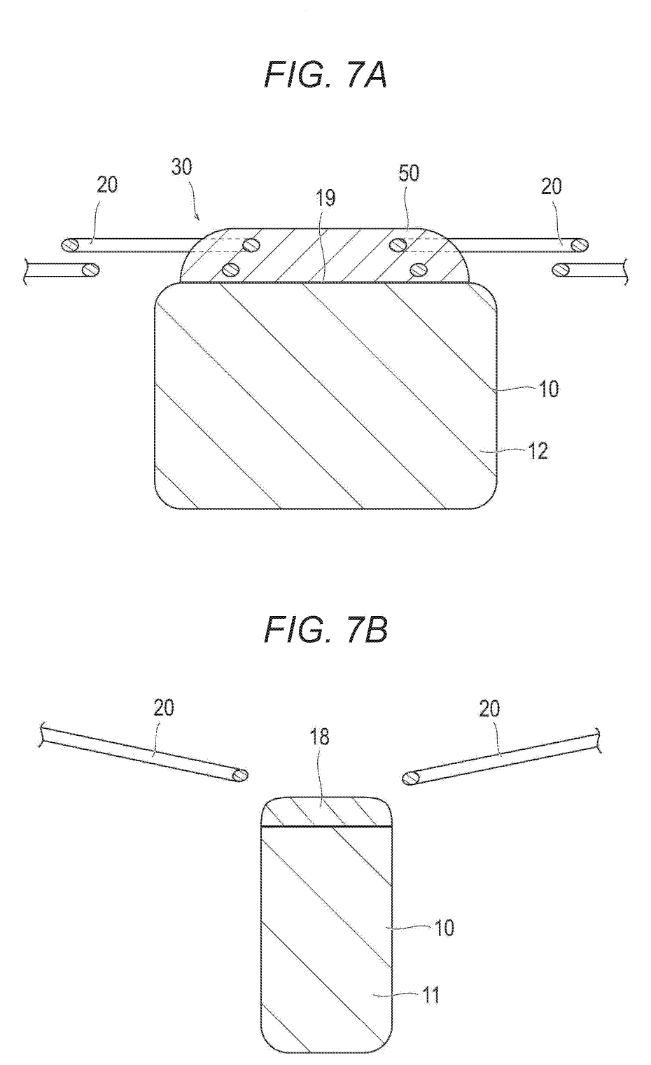

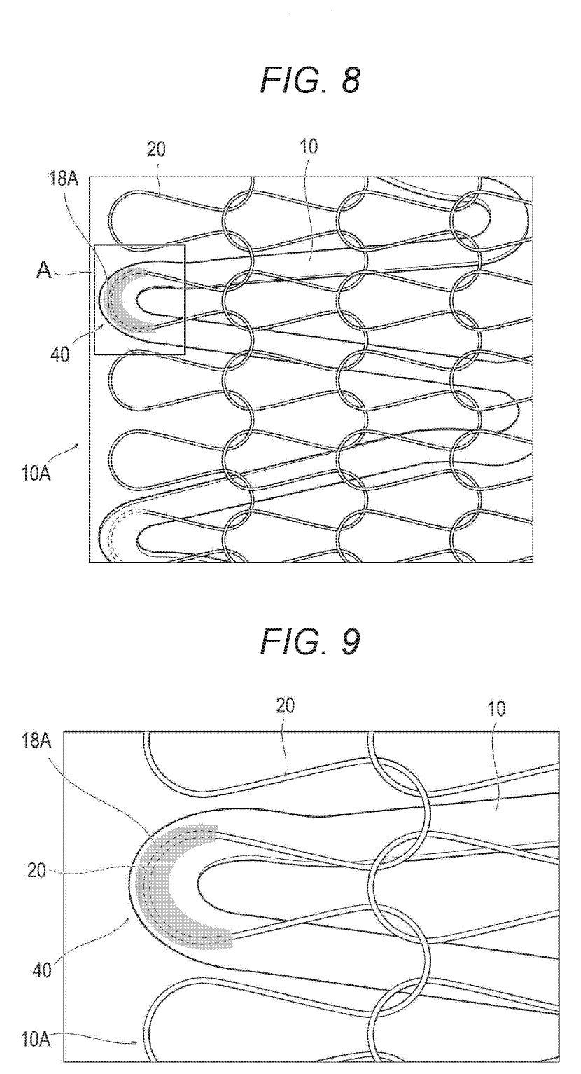

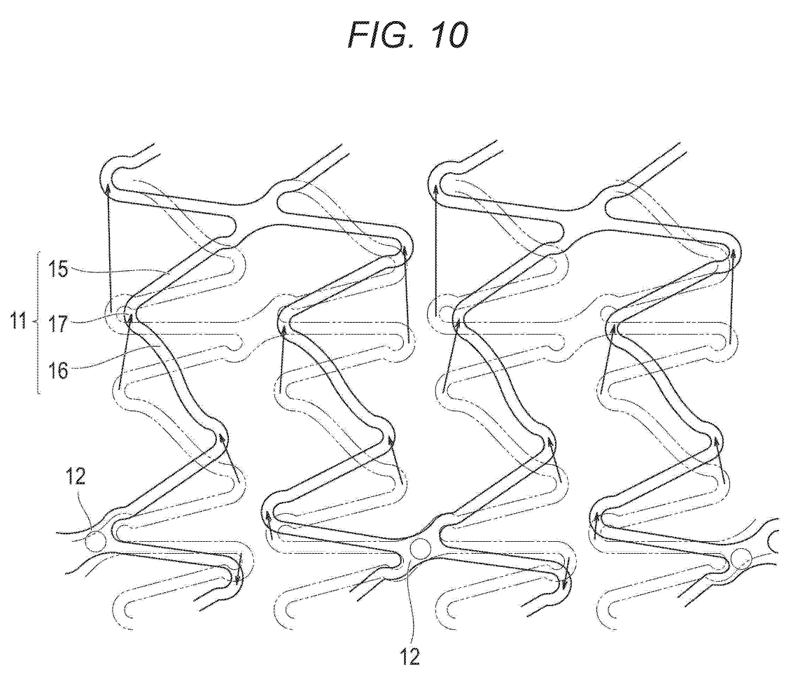

Hereinafter, a configuration of an implantable medical device 100 according to the present embodiment will be described with reference to FIGS. 1 to 10. FIG. 1 is a schematic plan view illustrating a stent delivery system 300 including the implantable medical device 100 according to an embodiment. FIG. 2 is a schematic plan view illustrating a contracted state of the implantable medical device 100 according to the present embodiment. FIG. 3 is a schematic plan view illustrating an expanded state of the implantable medical device 100 according to the present embodiment. FIG. 4 is a partially enlarged view illustrating the implantable medical device 100 according to the present embodiment. FIG. 5 is a partially enlarged view illustrating a portion A in FIG. 4. FIG. 6 is a partially enlarged view illustrating a portion A in FIG. 5. FIG. 7A is a cross-sectional view taken along a line 7A-7A in FIG. 6. FIG. 7B is a cross-sectional view taken along a line 7B-7B in FIG. 6. FIG. 8 is a schematic plan view illustrating the vicinity of a distal end of the implantable medical device 100 according to the present embodiment. FIG. 9 is a partially enlarged view illustrating a portion A in FIG. 8. FIG. 10 illustrates the shape of a stent 10 in a contracted state with a two-dot chain line, and illustrates the shape of the stent 10 in an expanded state with a solid line.

In the present specification, a horizontal direction in FIG. 2 will be referred to as an “axial direction” of the implantable medical device 100, and a vertical direction in FIG. 2 will be referred to as a “radial direction” of the implantable medical device 100. Furthermore, a side to be inserted into a living body will be referred to as a “distal side”, and a side which is opposite to the distal side and on which an operator such as a surgeon operates a medical device will be referred to as a “proximal side”.

As illustrated in FIG. 1, the stent delivery system 300 includes the implantable medical device 100 and a balloon catheter 200.

### Balloon Catheter 200

First, a configuration of the balloon catheter 200 will be described. The balloon catheter 200 is used to deliver the implantable medical device 100 in a contracted state to a lesion, expand the implantable medical device, and place the implantable medical device at the lesion.

The balloon catheter 200 includes an elongated catheter body portion 210, a balloon 220 provided at a distal end of the catheter body portion 210, and a hub 230 fixed to a proximal end of the catheter body portion 210.

The catheter body portion 210 includes an outer tube and an inner tube disposed inside the outer tube.

An expansion lumen through which an expansion fluid for expanding the balloon 220 flows is formed inside the outer tube. A distal end portion of the outer tube is fixed to a proximal end portion of the balloon 220. A proximal end portion of the outer tube is fixed to the hub 230.

A guidewire lumen into which a guidewire is to be inserted is formed inside the inner tube. A distal end portion of the inner tube penetrates the inside of the balloon 220 and opens on the distal end side of the balloon 220. A proximal end portion of the inner tube penetrates a side wall of the outer tube on the proximal end side of the balloon 220 and is fixed to the outer tube.

A material of the catheter body portion 210 is preferably a material having a certain degree of flexibility, and examples thereof include polyolefins such as polyethylene, polypropylene, polybutene, an ethylene-propylene copolymer, an ethylene-vinyl acetate copolymer, and an ionomer, or a mixture of two or more kinds thereof, thermoplastic resins such as a polyvinyl chloride resin, polyamide, a polyamide elastomer, polyester, a polyester elastomer, polyurethane, and a fluororesin, a silicone rubber, a latex rubber, and the like.

The balloon 220 is a member for expanding a stent such as the implantable medical device 100 described later and placing the stent at a target site. The distal end side of the balloon 220 is fixed to an outer wall surface of the inner tube. The proximal end side of the balloon 220 is fixed to an outer wall surface of the distal end portion of the outer tube. Thus, the inside of the balloon 220 communicates with the expansion lumen formed in the outer tube. The balloon 220 allows inflow of the expansion fluid from a proximal opening 231 through the expansion lumen. The balloon 220 is expanded or inflated due to the inflow of the expansion fluid, and is contracted or deflated and folded by the expansion fluid that has flowed therein being discharged.

A constituent material of the balloon 220 is preferably a flexible material that expands and contracts when the expansion fluid flows in and out, and examples thereof include polymer materials such as a polyolefin, a crosslinked polyolefin, polyester, a polyester elastomer, polyvinyl chloride, polyurethane, a polyurethane elastomer, polyphenylene sulfide, polyamide, a polyamide elastomer, and a fluororesin, a silicone rubber, a latex rubber, and the like. The constituent material of the balloon 220 is not limited to a mode in which the above-described polymer material is used alone, and a film in which the above-described polymer materials are appropriately laminated may be applied. Furthermore, the expansion fluid may be a gas or a liquid, and examples thereof include gases such as a helium gas, a CO2 gas and an O2 gas, and liquids such as physiological saline and an X-ray contrast agent.

The hub 230 includes the proximal opening 231 communicating with the expansion lumen of the outer tube. The proximal opening 231 functions as a port through which the expansion fluid flows in and out.

A constituent material of the hub 230 is not particularly limited, and examples thereof include thermoplastic resins such as polyethylene, polyurethane, polyester, polypropylene, polycarbonate, polyamide, polysulfone, polyarylate, and a methacrylate-butylene-styrene copolymer.

### Implantable medical device 100

Next, a configuration of the implantable medical device 100 will be described with reference to FIGS. 2 to 9. The implantable medical device 100 according to the present embodiment is used to treat a stenosed site or an occluded site generated in a blood vessel, a bile duct, a trachea, an esophagus, a urethra, or other body lumens. The implantable medical device 100 is a so-called balloon expandable type in which the implantable medical device is disposed on the folded balloon 220 in a crimped state, delivered to a lesion, and then expanded and placed in the lesion.

As illustrated in FIGS. 2 to 9, the implantable medical device 100 includes the stent 10 extending in the axial direction, a porous structure 20 disposed so as to cover an outer periphery of the stent 10, a fixing portion 30 at which the porous structure 20 is fixed to the stent 10, and both end fixing portions 40 at which the porous structure 20 is fixed to the stent 10 at a distal end 10A and a proximal end 10B of the stent 10.

The stent 10 extends in the axial direction (left and right direction in FIGS. 2 and 3) and includes the distal end 10A and the proximal end 10B. As illustrated in FIGS. 2 and 3, the stent 10 is formed so as to be capable of expanding in the radial direction (state illustrated in FIG. 3) and contracting (state illustrated in FIG. 2). As illustrated in FIGS. 3 to 6, the stent 10 includes wire-like rings 11 forming a cylindrical outer periphery at which gaps are formed, and link portions 12 connecting the rings 11 at the gaps.

As illustrated in FIG. 4, the wavy ring 11 includes a plurality of first strut portions 15 formed of straight or curved lines, a plurality of second strut portions 16 formed of straight or curved lines, and a plurality of curved portions 17 formed between the first strut portions 15 and the second strut portions 16.

The rings 11 are sequentially disposed side by side along the axial direction, and the rings 11 adjacent to each other in the axial direction are connected to each other by the link portions 12. It is therefore possible to easily obtain the stent 10 of a desired length by increasing or decreasing the number of rings 11.

A drug to be coated on an outer surface of the stent 10 is carried by a polymer to form a drug coating layer 18. The polymer is preferably a biodegradable polymer. In this case, after the stent 10 is implanted in the living body, the polymer is biodegraded while the drug is released in a sustained manner, so that restenosis at the stent implantation site is reliably prevented and inflammation derived from the polymer is suppressed (i.e., an anti-restenosis agent).

The biodegradable polymer is, for example, at least one polymer selected from the group consisting of polyester, aliphatic polyester, polyacid anhydride, polyorthoester, polycarbonate, polyphosphazene, polyphosphate ester, polyvinyl alcohol, polypeptide, polysaccharide, protein, and cellulose, a copolymer obtained by optionally copolymerizing monomers constituting the polymer, and a mixture of the polymer and/or the copolymer. Examples of the aliphatic polyester include polylactic acid (PLA), polyglycolic acid (PGA), and a lactic-glycolic acid copolymer (PLGA).

The drug coating layer 18 is disposed on the first strut portions 15 and the second strut portions 16 of the rings 11 illustrated in FIG. 4, and is disposed on the outer surface of the rings 11 as illustrated in FIG. 7B.

In other words, on the curved portions 17 and the link portions 12 of the rings 11 (sites where stress is concentrated and/or distortion occurs due to expansion deformation), the drug is not coated and the drug coating layer 18 is not formed, and thus, occurrence of stress concentration and/or distortion in the drug coating layer 18 is avoided even if the stent 10 is expanded.

Note that a primer coating layer (not illustrated) may be disposed between the drug coating layer 18 and the outer surface of the stent 10. A primer constituting the primer coating layer is selected in consideration of adhesion to the polymer contained in the drug coating layer 18 and adhesion to the outer surface of the stent 10, and the presence of the primer coating layer improves peel resistance of the drug coating layer 18.

The stent 10 is formed of a metal material or a polymer material. Here, the metal material used in a case where the stent 10 is formed of a metal material is not particularly limited, and a metal material usually used for the stent 10 can be used. Specific examples thereof include stainless steel such as SUS304, SUS316, SUS316L, SUS420J2, and SUS630, tantalum, titanium, a nickel-titanium alloy, a tantalum-titanium alloy, a nickel-aluminum alloy, Inconel, gold, platinum, iridium, tungsten, and cobalt-based alloys such as a cobalt-chromium (Co-Cr) alloy.

The polymer material used in a case where the stent 10 is formed of a polymer material is not particularly limited, and a polymer material usually used for the stent can be used. Specific examples thereof include polyolefins such as polyethylene and polypropylene, aromatic polyesters such as polyethylene terephthalate, cellulose-based polymers such as cellulose acetate and cellulose nitrate, and fluorine-containing polymers such as polytetrafluoroethylene and tetrafluoroethylene-ethylene copolymers.

The porous structure 20 is disposed so as to cover the outer periphery of the stent 10, and is expandable along with the expansion of the stent 10. By disposing the porous structure 20 on the outer periphery of the stent 10, the porous structure 20 is biased by the stent 10 against a blood vessel when the stent 10 is expanded in the blood vessel, so that detachment of the porous structure 20 due to a pressure of the blood flow flowing in the blood vessel is reduced. The porous structure 20 extends along the axial direction and has a knitted structure. Gaps of the knitted structure of the porous structure 20 are configured to be smaller than gaps of the adjacent rings 11. According to this configuration, when the stent 10 is implanted, peripheral emboli at the time of expansion of the stent 10 can be prevented. In addition, the porous structure 20 has a large number of penetrating voids or through-holes. A size of the void is preferably smaller in area than the gap between the rings 11 of the stent 10. This makes it possible to prevent scattering of plaque and thrombus associated with expansion of the stent 10. On the other hand, the size of the void is preferably larger than an area of a single blood cell contained in the blood. This allows blood cells to pass through the voids. Furthermore, a large number of penetrating voids impart a stretchable property to the porous structure 20, and thus, when the stent 10 is expanded in the radial direction, the porous structure 20 also extends in the circumferential direction and easily follows the expansion of the stent 10.

The porous structure 20 includes a knitted structure (knit), a woven structure (braid), and a molded structure. In a case where the porous structure 20 is a knitted structure, the porous structure 20 is formed of a stockinette stitch. The stockinette stitch is a known knitting method, and thus, detailed description thereof will be omitted. By configuring the porous structure 20 by stockinette stitch, it is possible to suppress shortening of the porous structure 20 in the axial direction due to expansion of the stent 10. In a case where the porous structure 20 is a woven structure, the woven structure is formed by a known weaving method. The porous structure 20 may be configured with a molded structure in which a hole is formed in a cylindrical object formed by injection molding, or the like. The size, shape, and number of meshes and weaves of the porous structure 20 are not particularly limited as long as peripheral emboli at the time of expansion of the stent 10 can be prevented.

The material constituting the porous structure 20 is not particularly limited, but examples thereof include (1) a polymer selected from the group consisting of aliphatic polyester, polyester, polyacid anhydride, polyorthoester, polycarbonate, polyphosphazene, polyphosphoric acid ester, polyvinyl alcohol, polypeptide, polysaccharide, protein, and cellulose.; (2) a copolymer composed of two or more monomers constituting the (1). Here, the aliphatic polyester is not particularly limited, and examples thereof include polylactic acid (PLA) such as poly-L-lactic acid (PLLA), poly-D-lactic acid (PDLA), and poly-DL-lactic acid (PDLLA), polyglycolic acid (PGA), polyhydroxybutyric acid, polyhydroxyvaleric acid, polyhydroxypentanoic acid, polyhydroxyhexanoic acid, polyhydroxyheptanoic acid, poly (ε-caprolactone) (PCL), polytrimethylene carbonate, poly 2,2-dimethyltrimethylene carbonate, polydioxanone, polybutyrolactone, polyvalerolactone, polymalic acid, polyethylene adipate, polyethylene succinate, polybutylene adipate, and polybutylene succinate. The polycarbonate is not particularly limited, and examples thereof include tyrosine-polycarbonate. Further, polymers other than the biodegradable polymers described above can also be used.

In addition, the porous structure 20 may be formed of a metal material, and in that case, the same material as the metal material exemplified in the stent 10 described above can be used.

A yarn diameter of the porous structure 20 is not particularly limited, but can be, for example, 20 μm.

In the fixing portion 30, generally, at least part of the porous structure 20 is fixed to the stent 10 at a position excluding the distal end 10A and the proximal end 10B of the stent 10. In the present specification, the distal end 10A of the stent 10 is defined as a “region that is the most distal end of the stent 10 and is not provided with the link portion 12 disposed at the most distal end”. Furthermore, in the present specification, the proximal end 10B of the stent 10 is defined as a “region that is the most proximal end of the stent 10 and is not provided with the link portion 12 disposed at the most proximal end”.

The fixing portion 30 is a portion at which the porous structure 20 is fixed to the stent 10. In the present embodiment, the fixing portion 30 is formed of a weldable fixing member 50. In the fixing portion 30, the porous structure 20 is embedded in the fixing member 50 by the fixing member 50 being welded, and the porous structure 20 is fixed to the stent 10.

As a material to be used for the fixing member 50, the polymer material exemplified as the material constituting the porous structure 20 described above can be used. Further, paraffin may be used as the fixing member 50.

In the present embodiment, as illustrated in FIGS. 3 to 6, the fixing portion 30 is provided at all of the link portions 12.

In the implantable medical device 100 according to the present embodiment, as illustrated in FIGS. 6 and 7A, the fixing portion 30 is provided at the link portion 12. Here, as illustrated in FIG. 10, the link portion 12 is a portion at which a change in axial geometry change accompanying the expansion of the stent 10 is small. Thus, tension in the porous structure 20 due to expansion of the stent 10 does not occur, and peeling or breakage of the porous structure 20 can be suitably suppressed.

Further, as described above, the link portion 12 is not provided with the drug coating layer 18. Thus, as illustrated in FIG. 7A, the fixing portion 30 is configured such that the porous structure 20 is directly fixed to the stent 10 in the exposed portion 19 of the stent 10 to which a drug is not applied. In other words, as illustrated in FIG. 7A, the fixing member 50 constituting the fixing portion 30 is directly fixed to the link portion 12 of the stent 10. According to this configuration, as compared with a configuration in which the fixing member 50 constituting the fixing portion 30 is fixed to the stent 10 via the drug coating layer 18, adhesive strength of the porous structure 20 to the stent 10 when the fixing member 50 is welded can be improved.

The fixing portion 30 is configured such that the porous structure 20 is directly fixed to the stent 10 in the exposed portion 19 of the stent 10 to which a drug is not applied. Thus, even if the fixing portion 30 is formed by heating such as laser irradiation, the drug coating layer 18 is not present, and thus, the efficacy of the drug is not lost. Note that the configuration in which the fixing member 50 is fixed to the stent 10 via the drug coating layer 18 can be adopted in a case where the fixing portion 30 is formed by a method without heating or with little influence of heating such as an adhesive.

As illustrated in FIG. 6, a loop portion 21 that is knitted along the circumferential direction (vertical direction in FIG. 6) of the porous structure 20 is configured to be fixed to the stent 10 at the fixing portion 30. The portion to be fixed includes at least the loop portion 21, and may include an intersection with an adjacent stitch. According to this configuration, the loop portion 21 extending along the circumferential direction is fixed to the stent 10, and thus, a long distance of the porous structure 20 can be embedded in the fixing member 50, and the fixing force is improved.

As illustrated in FIGS. 8 and 9, at the both end fixing portions 40, the porous structure 20 is fixed to the stent 10 at the distal end 10A and the proximal end 10B of the stent 10. In particular, as illustrated in FIGS. 8 and 9, the both end fixing portions 40 are preferably provided at a curved portion 18A at the distal end 10A of the stent 10 and a curved portion (not illustrated) at the proximal end 10B of the stent 10.

A method of fixing the porous structure 20 to the stent 10 in the both end fixing portions 40 is the same as the method of fixing the porous structure 20 to the stent 10 in the fixing portion 30 described above, and thus, the description thereof will be omitted.

The implantable medical device 100 according to the present embodiment has the both end fixing portions 40, and thus, when the implantable medical device 100 is delivered to the lesion, it is possible to prevent the porous structure 20 from being curled from the stent 10 even in a case where the curvature to the lesion is strong.

As described above, the implantable medical device 100 according to the present embodiment includes the stent 10 extending in the axial direction, including the distal end 10A and the proximal end 10B, formed expandable in the radial direction, and including the plurality of wavy rings 11 forming the cylindrical outer periphery, and the link portion 12 connecting the adjacent rings 11, the porous structure 20 disposed to cover the stent 10 and having a large number of penetrating voids, and the fixing portion 30 at which at least part of the porous structure 20 is fixed to the stent 10 at a position other than the distal end 10A and the proximal end 10B. According to the implantable medical device 100 configured as described above, at least part of the porous structure 20 is fixed to the stent 10 at a position other than the distal end 10A and the proximal end 10B by the fixing portion 30, so that it is possible to suppress the porous structure 20 from being deformed so as to spread radially outward and the outer diameter of the entire implantable medical device 100 from becoming large. It is therefore possible to prevent the porous structure 20 from being deformed so as to spread radially outward and deliverability of the implantable medical device 100 from being lowered.

Further, the fixing portion 30 is provided at the link portion 12. According to the implantable medical device 100 configured as described above, peeling or breakage of the porous structure 20 can be suitably suppressed.

In addition, the fixing portion 30 is configured such that the porous structure 20 is directly fixed to the stent 10 at the exposed portion 19 of the stent 10 to which a drug is not applied. According to the implantable medical device 100 configured as described above, the adhesive strength when the fixing member 50 is welded can be improved as compared with a configuration in which the fixing member 50 constituting the fixing portion 30 is fixed to the stent 10 via the drug coating layer 18.

In addition, the implantable medical device 100 further includes both end fixing portions 40 in which the porous structure 20 is fixed to the stent 10 at the distal end 10A and the proximal end 10B. According to the implantable medical device 100 configured as described above, when the implantable medical device 100 is delivered to the lesion, it is possible to prevent the porous structure 20 from being curled from the stent 10 even in a case where the curvature to the lesion is strong.

In addition, at the fixing portions 30, the stent 10 and the porous structure 20 are fixed by welding of the biodegradable polymer. According to the implantable medical device 100 configured as described above, the fixing portion 30 is biodegraded, and thus, polymer-derived inflammation is suppressed.

The porous structure 20 is formed of a stockinette stitch, and the fixing portion 30 is configured by fixing the loop portion 21 of meshes knitted along the circumferential direction of the porous structure 20 to the stent 10. According to the implantable medical device 100 configured as described above, the loop portion 21 knitted along the circumferential direction is fixed to the stent 10, and thus, a long distance of the porous structure 20 can be embedded in the fixing member 50, and the fixing force is improved.

Although the configuration of the implantable medical device 100 according to the present invention has been described above through the embodiment, the present invention is not limited to the configuration described in the embodiment, and can be appropriately changed based on the description of the claims.

For example, in the above-described embodiment, the fixing portion 30 is provided at all of the link portions 12, but the fixing portion 30 may be provided at some of all the link portions 12. According to this configuration, it is possible to suppress the porous structure 20 from being deformed so as to expand radially outward and the outer diameter from becoming large while reducing a usage amount of the fixing member 50 constituting the fixing portion 30.

In the embodiment described above, the fixing portion 30 is provided at the link portion 12. However, the fixing portion may be provided at the first strut portion 15 or the second strut portion 16. Also with this configuration, it is possible to prevent the porous structure 20 from being deformed so as to spread radially outward, which would reduce the deliverability of the implantable medical device.

In the embodiment described above, the fixing portion 30 is provided at the link portion 12. However, the fixing portion may be provided at the link portion and the strut. According to this configuration, the fixing portion is provided at the link portion and the strut, and thus, the strength is further improved.

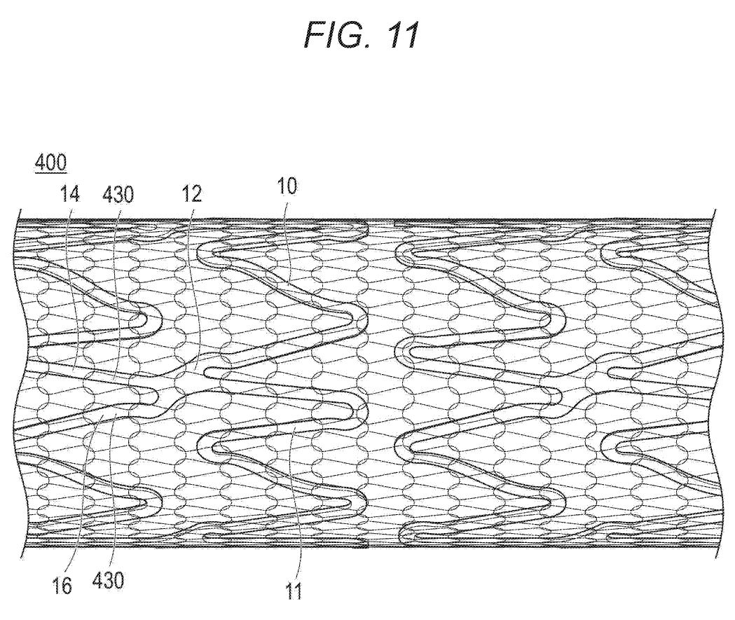

In the embodiment described above, the fixing portion 30 is provided at the link portion 12. However, as illustrated in FIG. 11, a fixing portion 430 of the implantable medical device 400 according to the modification may be provided at a third strut portion (first strut) 14 and/or a second strut portion (second strut) 16 which are adjacent to the link portion 12 on one side (for example, the left side in FIG. 11) in the axial direction and provided in pairs in the circumferential direction (vertical direction in FIG. 11). Note that FIG. 11 illustrates, as an example, a form in which the fixing portion 430 is provided at the third strut portion 14 and the second strut portion 16. According to this configuration, the third strut portion 14 and the second strut portion 16 are provided near the link portion 12, and thus, a change in geometry in the axial direction before and after expansion is small as compared with other struts, and peeling or breakage of the porous structure 20 can be reduced. In addition, according to this configuration, as compared with the configuration in which the fixing portion exists at the first strut portion 15 or the second strut portion 16, a possibility of greatly opening a space between the first strut portion 15 and the next first strut portion 15 separated in the radial direction is reduced, so that peeling or breakage of the porous structure 20 can be reduced. In addition, by providing the fixing portion 430 at the third strut portion 14 or the second strut portion 16, peeling or breakage of the porous structure 20 can be further reduced as compared with the case where the fixing portion 430 is provided at both the third strut portion 14 and the second strut portion 16.

In the embodiment described above, the stent 10 and the porous structure 20 are fixed by welding of the biodegradable polymer at the fixing portion 30. However, a method of fixing the porous structure 20 to the stent 10 is not limited, and polymer welding other than a biodegradable polymer may be used. Furthermore, the method may be adhesive fixing by an adhesive, binding fixing by a binding band, clip fixing, or the like.

Furthermore, in the embodiment described above, the implantable medical device 100 is a balloon-expandable stent. However, the implantable medical device 100 may be a so-called self-expanding stent that is compressed in the central axis direction at the time of in vivo insertion, and expands outward to restore the shape before compression at the time of in vivo implantation. In a case where the stent 10 is a self-expanding type, a superelastic alloy such as a nickel-titanium alloy, or the like, is preferable because a restoring force to the original shape is required.

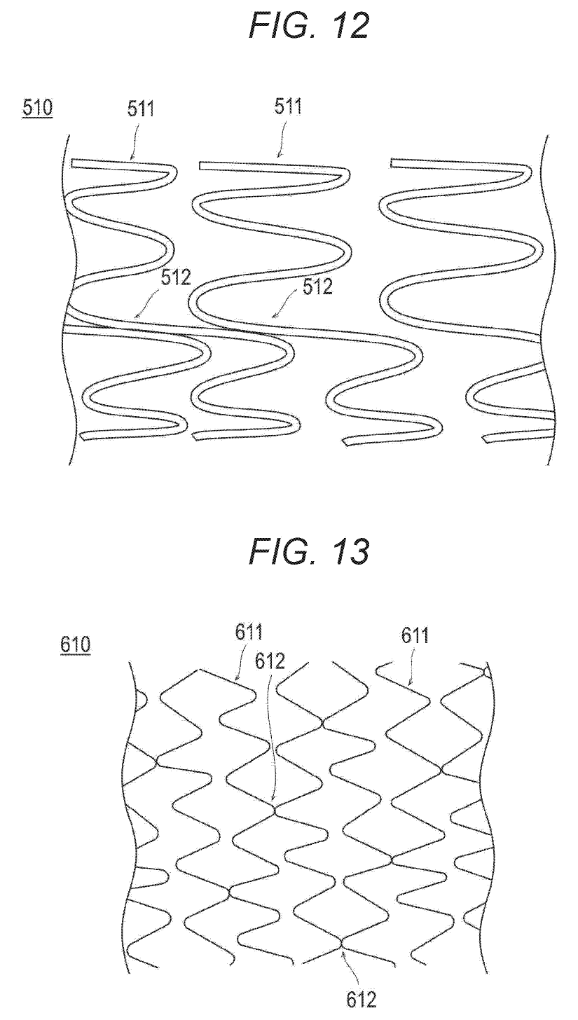

In addition, the shape of the stent is not particularly limited, and the stent may have a shape as illustrated in FIGS. 12 and 13. For example, a stent 510 illustrated in FIG. 12 includes rings 511 and link portions 512. In addition, a stent 610 illustrated in FIG. 13 includes rings 611 and link portions 612.

Further, in the above-described embodiment, the fixing member 50 is melted by being irradiated with a laser, but the fixing member 50 may be melted by being irradiated with a light beam or an ultrasonic wave or by the entire being heated.

What is claimed is:

1. An implantable medical device comprising:
a stent that has a cylindrical shape extending in an axial direction, is expandable in a radial direction, and includes:
a plurality of wavy rings forming an outer periphery of the cylindrical shape, and
a plurality of link portions each connecting two of the rings that are adjacent to each other;
a porous structure that covers the stent and has a plurality of through-holes; and
a plurality of first fixing portions by which at least part of the porous structure is fixed to the stent, each of the first fixing portions being located on the stent at a position other than distal and proximal ends of the stent.

2. The implantable medical device according to claim 1, wherein each of the first fixing portions is located on one of the link portions.

3. The implantable medical device according to claim 1, wherein
each of the wavy rings includes a first strut and a second strut that are adjacent to each other in a circumferential direction and adjacent to one of the link portions, and
each of the first fixing portions is located on either the first strut or the second strut of one of the wavy rings.

4. The implantable medical device according to claim 1, further comprising:
a drug coating layer that partly covers an outer surface of the stent, wherein
the first fixing portions are located at portions of the stent that are not covered by the drug coating layer.

5. The implantable medical device according to claim 4, wherein the drug coating layer is formed of an anti-restenosis agent.

6. The implantable medical device according to claim 1, further comprising:
a pair of second fixing portions by which the porous structure is fixed to the stent at the distal and proximal ends of the stent.

7. The implantable medical device according to claim 6, wherein
each of the wavy rings located at the distal and proximal ends of the stent includes a curved portion, and
the pair of second fixing portions are located on the curved portions of the wavy rings at the distal and proximal ends of the stent.

8. The implantable medical device according to claim 1, wherein the porous structure is fixed to an outer periphery of the stent.

9. The implantable medical device according to claim 1, wherein the first fixing portions are made of a polymer.

10. The implantable medical device according to claim 9, wherein the first fixing portions are made of a biodegradable polymer welded to fix the stent to the porous structure.

11. The implantable medical device according to claim 1, wherein the first fixing portions are made of paraffin.

12. The implantable medical device according to claim 1, wherein the porous structure includes thread-like members that are knitted or woven to form the through-holes between the thread-like members.

13. The implantable medical device according to claim 12, wherein the porous structure is a mesh having a knitted structure that is expandable along with expansion of the stent.

14. The implantable medical device according to claim 1, wherein
the porous structure is formed of a stockinette stitch, and
each of the first fixing portions includes a loop portion knitted along a circumferential direction of the porous structure, the loop portion being fixed to the stent.

15. The implantable medical device according to claim 1, wherein an area of each of the through-holes is smaller than an area of a gap between two of the wavy rings that are adjacent to each other.

16. A stent delivery system comprising:
a balloon catheter including an elongated catheter body portion and a balloon at a distal end of the catheter body portion; and
an implantable medical device disposed on the balloon and including:
a stent that has a cylindrical shape extending in an axial direction, is expandable in a radial direction, and includes:
a plurality of wavy rings forming an outer periphery of the cylindrical shape, and
a plurality of link portions each connecting two of the rings that are adjacent to each other,
a porous structure that covers the stent and has a plurality of through-holes, and
a plurality of first fixing portions by which at least part of the porous structure is fixed to the stent, each of the first fixing portions being located on the stent at a position other than distal and proximal ends of the stent.

17. The stent delivery system according to claim 16, wherein
the catheter body portion includes:
an outer tube having a lumen that communicates with an interior of the balloon, and
an inner tube inside the outer tube, the inner tube having a guidewire lumen and extending through the interior of the balloon.

18. The stent delivery system according to claim 16, wherein each of the first fixing portions is located on one of the link portions.

19. The stent delivery system according to claim 16, wherein
each of the wavy rings includes a first strut and a second strut that are adjacent to each other in a circumferential direction and adjacent to one of the link portions, and
each of the first fixing portions is located on either the first strut or the second strut of one of the wavy rings.

20. The stent delivery system according to claim 16, wherein
the implantable medical device further includes a drug coating layer that partly covers an outer surface of the stent, and
the first fixing portions are located at portions of the stent that are not covered by the drug coating layer.
